(19) United States
(12) Patent Application Publication
(10) Pub. No.: US 2024/0268278 A1
(43) Pub. Date: Aug. 15, 2024

(54) SYSTEM AND METHOD FOR MONITORING POLLINATION OF PLANTS

(71) Applicant: SIGNIFY HOLDING B.V., EINDHOVEN (NL)

(72) Inventors: CÉLINE CATHERINE SARAH NICOLE, EINDHOVEN (NL); MARC ANDRE DE SAMBER, LOMMEL (BE); HARRY BROERS, 'S-HERTOGENBOSCH (NL); RICHARD LUDWIG EDUARD VAN HAASEN, EINDHOVEN (NL)

(21) Appl. No.: 18/567,938

(22) PCT Filed: Jun. 7, 2022
(86) PCT No.: PCT/EP2022/065329

§ 371 Date: Dec. 7, 2023

(30) Foreign Application Priority Data
Jun. 8, 2021 (EP) 21178297.4

(51) Int. Cl. A01G 7/00

(52) U.S. Cl. CPC A01G 7/00

(57) ABSTRACT

A system is disclosed for monitoring pollination of plants carrying one or more flowers. The system comprises a plurality of microphones for monitoring an area comprising said one or more flowers. Each microphone out of the plurality of microphones is configured to monitor a sub-area of the monitored area. Further, each microphone is suitable for recording sounds produced by a pollinator, such as a bumblebee, that is present in the microphone's sub-area and is configured to output one or more signals indicative of recorded sounds. The system further comprises a data processing system that is configured to receive, from each of the plurality of microphones, said one or more signals indicative of recorded sounds. The data processing system is further configured to determine, based on the signals received from the plurality of microphones, a value of a pollination quality parameter indicative of how well one or more flowers in the monitored area are pollinated.

### FIELD OF THE INVENTION

This disclosure relates to a system for monitoring pollination of plants carrying one or more flowers, in particular to such system that comprises a plurality of microphones for monitoring an area comprising the one or more flowers. This disclosure further relates to a corresponding method for monitoring pollination of plants as well as to a data processing system, computer-readable storage medium and computer program for performing such method.

### BACKGROUND

The fertilization in a plant is a process of sexual reproduction, which occurs after pollination and germination. It is defined as the fusion of the male gametes (pollen) with the female gametes (ovum) to form a diploid zygote. Fertilization is a physicochemical process which occurs after the pollination of the carpel. The complete series of this process takes place in the zygote to develop into a seed.

In the fertilization process, flowers play a significant role as they are the reproductive structures of angiosperms (flowering plants). The method of fertilization in plants occurs when gametes in haploid conditions fuse to produce a diploid zygote.

Pollination, during which the transfer of pollen from a male part of a plant to a female part of a plant happens, is essential in fertilisation and the production of seeds and fruits the seeds reside in. Hence, pollination is very important in the horticulture industry. If pollination does not happen effectively for a batch of plants, then the yield of this batch will drop significantly. Pollination can happen via passive (e.g. wind) or active mechanisms. Pollinators, such as bumble bees, honeybees, birds, bats, butterflies, flower beetles, etc. are active contributors to pollination. A pollinator may be understood to be an animal that moves pollen from a male anther of a flower to the female stigma of a flower. In greenhouses for example, bumblebees are typically used to pollinate the plants. A grower can purchase the bees, which arrive in a box, and position the box at an appropriate position within the greenhouse. Then, the bees will fly from flower to flower and perform their pollination function. Advantages of such natural pollination techniques is that they provide for efficient pollination, at least more efficient in comparison with techniques using human labour.

A disadvantage of such natural pollination process is that a greenhouse operator has only limited control over it, i.e. has only limited control over the behaviour of the pollinators. The pollinators, such as bumblebees, may namely avoid locations in the greenhouse at which circumstances are less attractive for them. If at some region in the greenhouse, the temperature is for example relatively low and/or undesired air flows are present and/or sub-optimal lighting is present, then the pollinators may avoid this region, meaning that the flowers in this region will not be pollinated very well. The consequence may be that the plants in this region will have a very low fruit yield.

Hence, it is very important that the greenhouse operator can monitor how well the pollinators are pollinating the flowers throughout the greenhouse. Only then can the greenhouse operator take adequate actions if pollination does not happen satisfactorily somewhere in the greenhouse. Thus, there is a need in the art for a system and method for monitoring pollination of plants.

### SUMMARY

To that end a system is disclosed for monitoring pollination of plants carrying one or more flowers. The system comprises a plurality of microphones for monitoring an area comprising said one or more flowers. Each microphone out of the plurality of microphones is configured to monitor a sub-area of the monitored area. Further, each microphone is suitable for recording sounds produced by a pollinator, such as a bumblebee, that is present in the microphone's sub-area and is configured to output one or more signals indicative of recorded sounds. The system further comprises a data processing system that is configured to receive, from each of the plurality of microphones, said one or more signals indicative of recorded sounds. The data processing system is further configured to determine, based on the signals received from the plurality of microphones, a value of a pollination quality parameter indicative of how well one or more flowers in the monitored area are pollinated.

With the system, the pollination of plants can be efficiently monitored. The system can be easily installed and does not require substantial adaptations to other apparatuses in the greenhouse. The microphones need only be appropriately positioned in the greenhouse, preferably such that they together cover a substantial part of the greenhouse. The microphones can for example be installed in already present and/or nearby luminaires in a greenhouse. The data processing system may be present outside of the greenhouse. In an example, the data processing system is a desktop computer (having installed an appropriate computer program) in the office of the greenhouse operator. Advantageously, a microphone is a relatively simple apparatus that can still monitor a relatively large sub-area, not least because it can record sounds from pollinators that are for example behind some leaves as viewed from the position of the microphone. This in contrast to for example imaging systems which would not record a pollinator if it is not in sight. The recorded sounds, in case pollinators are used for pollinating the flowers in the greenhouse, will also indicate sounds associated with the pollination of a flower by a pollinator. Hence, based on the recorded sounds, the data processing system can determine one or more pollination quality parameters described herein. In principle, the more sounds associated with pollination are indicated by the one or more signals received from the microphones, the higher the value of the pollination quality parameter (assuming that a higher value of the pollination quality parameter corresponds to better pollination).

The one or more signals may be indicative of recorded sounds in that they are indicative of one or more audiograms representing the recorded sounds. An audiogram may be understood to refer to a frequency spectrum of a sound, i.e. a spectrum indicating an intensity, e.g. expressed in dB, per frequency or per frequency bandwidth.

In principle, the better the pollination of flowers in the monitored area, the higher the yield will be. One batch of flowers may be better pollinated than another batch of flowers in the sense that the flowers in said one batch are on average pollinated more times, i.e. are on average visited more often by pollinators, than the flowers in the other batch and/or in the sense that the flowers in said one batch are more effectively pollinated during a pollination event than the flowers in the other batch.

Any kind of plant may be monitored with the disclosed system, for example fruit plants, such as grapevines, blueberry plants, strawberry plants, raspberry plants, blackberry plants, apple trees, cherry trees, peach trees, et cetera, and vegetable plants, such as cucumber plants, tomato plants, eggplants, pepper plants, et cetera.

Some or all of the microphones may be configured to record sounds produced by a pollinator in a sub-area surrounding the microphone entirely, i.e. the microphones may be 360 degrees microphones. Additionally or alternatively, the microphones may be directional in that they are configured to monitor only a specific part of their surrounding environment. A microphone may be configured to monitor a part of at least one plant, e.g. a part of a first plant and a part of a second plant. In such case, the sub-area of the microphone comprises only a part of at least one plant. To illustrate, a microphone may be configured to monitor the top part of a high wire tomato plant. Alternatively, a microphone may be configured to monitor multiple plants, as would for example typically be the case with strawberry plants. In such case, the sub-area of the microphone comprises several plants. The sub-areas of the respective microphones may or may not overlap.

Examples of pollinators are bumblebees, honeybees, birds, such as hummingbirds, bats, butterflies, flower beetles, etc.

The one or more signals that each microphone is configured to output may be referred to as sound signals because they are indicated of recorded sounds. Preferably, the one or more sounds signals comprise an identifier of the microphone that sends them so that the data processing system is able to determine from which microphone the signals originate. Further, the data processing system preferably has stored the respective identifiers of the microphones in association with their respective sub-areas in the greenhouse that they monitor. This enables the data processing system to determine in which sub-areas in the greenhouse the sounds are produced as indicated by the one or more signals from the microphones.

As stated, the data processing system is configured to determine a value of a pollination quality parameter indicative of how well one or more flowers in the monitored area are pollinated. It should be appreciated that this may be embodied as the data processing system being configured to determine a pollination quality parameter indicative of how well one or more flowers in a specific region of the monitored area are pollinated. The data processing system may be configured to determine several pollination quality parameters respectively indicative of how well one or more flowers in several regions of the monitored area are pollinated. The data processing system being configured to determine a value of a pollination quality parameter indicative of how well one or more flowers in the monitored area are pollinated may additionally or alternatively be embodied as the data processing system being configured to determine a combined pollination quality parameter based on several pollination quality parameters associated with several regions of the monitored area.

In an embodiment, the data processing system is configured to determine, based on the one or more signals received from the plurality of microphones, a number of pollination events in the monitored area or in a region of the monitored area and/or a duration of each of the pollination events in the monitored area or in a region of the monitored area, wherein each pollination event comprises a pollinator visiting a flower. A pollination quality parameter can then be based on a number and/or duration of determine pollination events. In such embodiment, a value of the pollination quality parameter can be determined based on the number of pollination events and/or based on the duration of each of the pollination events. This embodiment provides a convenient manner for determining the pollination quality parameter. The number of pollination events and their respective durations is positively correlated with how well flowers are pollinated.

In an embodiment, the data processing system is configured to determine a pollination event by determining a probability that a pollination event occurred and counting it as a pollination event if the determined probability is higher than a threshold probability, e.g. higher than 50%.

It should be appreciated that a pollinator visiting a flower does not necessarily mean that a pollination event occurs, for example when the flower requires sonication to release pollen and a sonication sound or sonication pattern turns out not to be effective or sufficient in terms of energy (provided by the pollinator to the flower) or duration. In such case, is may be that the data processing system determines that a pollinator has visited a flower, yet that the data processing system does not recognize a sound and/or sound pattern that is associated with a pollination event.

In an embodiment, the data processing system is configured to determine for one or more of the determined pollination events, of which species or insect type the pollinator is. This is advantageous in that it allows to track the diversity and other characteristics of the pollinator population. If many pollinations are done by wild honeybees that got into the green house by “accident” for example, maybe it is not needed to add an additional hive of bumblebees.

In an embodiment, the data processing system is configured to determine, based on the one or more signals received from the plurality of microphones, for each region out of a plurality of regions in the monitored area, a pollination quality parameter indicative of how well one or more flowers in the region are pollinated. This embodiment advantageously allows to monitor the quality of the pollination per region of the total area as monitored by the plurality of microphones and thus enables a greenhouse operator to see at which locations the pollination quality is relatively low.

The value of the pollination quality parameter, especially if it is determined sequentially for the same region, may also be used for forecasting when the plants in that region are ready for harvest. Thus, in an embodiment, the data processing system is configured to determine, based on the determined value for pollination quality parameter, a harvest time at which produce is ready for harvest. More in particular, since the data processing system may be configured to determine a pollination quality parameter value per region in the monitored area, the data processing system may be configured to determine such harvest time per region of the monitored area.

In an embodiment, the data processing system is configured to, for each region out of a plurality of regions in the monitored area, determine, based on the one or more signals received from the plurality of microphones, a number of pollination events in the region and/or a duration of each of the pollination events in the region, wherein each pollination event comprises a pollinator visiting a flower. In this embodiment, the data processing is further configured to, for each region, based on the determined number of pollination events in the region and/or based on the determined durations of the pollination events in the region, determine a value of a pollination quality parameter indicative of how well one or more flowers in the region are pollinated. This embodiment enables to determine the pollination quality per region in a convenient manner.

The monitored area comprises several regions. The regions may be the same as the sub-areas. In such case, a pollination quality parameter is determined for each sub-area, thus effectively one for each microphone. However, this does not need to be the case per se. The regions for which pollination quality parameters are determined may be different from the sub-areas. To illustrate, it may very well be that only few pollination events happen specifically in a region where the two sub-areas of two respective neighboring microphones overlap. Then, a low pollination quality parameter may be determined for this specific region, whereas higher pollination quality parameters may be determined for other regions in the two sub-areas of the two respective microphones.

The data processing system may be configured to determine a number of pollination events in the sense that it is configured to determine a number of pollination events per unit of time, e.g. per week, per day, per hour, per minute, et cetera.

In an embodiment, the data processing system is configured to determine a pollination event by recognizing from the one or more signals from the plurality of microphones, a sound that is associated with a pollination event and/or a sound pattern that is associated with a pollination event. This embodiment enables to accurately determine pollination events and, potentially, their respective durations.

The sound may be recognizable in that it has a certain frequency. The sound pattern can be a time-lapsed sound pattern and/or a frequency pattern, such as a Fast Fourier Transform (FFT) pattern.

Preferably, the data processing system has stored one or more reference sounds and/or reference sound patterns that are associated with respective one or more pollination events. A reference sound may be obtained by recording the sound that a pollinator produces when it actually pollinates a flower. In any case it should be confirmed that pollination occurs when the reference sound is recorded. This may be performed simply by a human observer who can indicate at what time a pollination event has occurred and was effective. A reference time-lapsed sound pattern can be obtained similarly. A reference frequency pattern can be obtained by Fourier transforming a recorded reference sound.

For this embodiment, in principle any sound classification technique, also referred to as audio classification technique, known in the art can be used for recognizing the sound and/or sound pattern associated with a pollination event. Neural network can for example be used as described in Applying Neural Network on the Content-Based Audio Classification by Xi Shao et al, Fourth International Conference on Information, Communications and Signal Processing, 2003 and the Fourth Pacific Rim Conference on Multimedia. Proceedings of the 2003 Joint, 15-18 Dec. 2003. The training data set referred to in this publication may then be formed by recorded reference sounds and/or reference sound patterns.

Predicting species identity of bumblebees through analysis of flight buzzing sounds by Gradišek et al, The International Journal of Animal Sound and its Recording, Volume 26, 2017—Issue 1, pages 63-76, May 2016 discloses an example spectrograph for different types of buzzing, including sonication, for B. hypnorum worker as well as sound classification techniques that can be used for recognizing sounds associated with a pollination event.

In an embodiment, the sound pattern is a time-lapsed sound pattern and comprises a first time period comprising a sound associated with flying of a pollinator, a subsequent second time period substantially without sounds associated with flying of a pollinator and a subsequent third time period comprising a sound associated with flying of a pollinator.

Typically, during pollination, the pollinator does not fly. Hence, a non-flying period in between two flying periods may indicate a pollination event.

The sound or sound pattern should be recognized in noisy environments. It may very well be that the recorded sounds contain sounds of other flying pollinators during the second time period. However, in terms of recognizing this pollination event, such flying sound of other pollinators is to be regarded as noise.

In an embodiment, the data processing system is configured to determine a duration of a pollination event based on a duration of the second time period in the pattern as recognized in the recorded sounds.

In an embodiment, the pollinators are bees, such as bumblebees and the sound associated with a pollination event is a sonication sound and/or the sound pattern associated with a pollination event is a sonication sound pattern. Bees are known to make use of vibrations to extract pollen from anthers. This is referred to as sonication and also as buzz-pollination. During sonication, the bees produce a sonication sound. The characteristics of sonication sound is described in detail in What's the ‘buzz’ about? The ecology and evolutionary significance of buzz-pollination by Paul De Luca and Mario Vallejo-Marín, Current Opinion in Plant Biology 2013, 16:429-435.

A sonication sound may be understood to refer to a sound that is produced by a bee that is sonicating and a sonication sound pattern may be understood to refer to a frequency sound pattern that is produced by a bee that is sonicating.

In an embodiment, the data processing system is configured to determine a duration of a pollination event based on a duration of the sonication sound.

In an embodiment, the plurality of microphones comprises a first microphone configured to monitor a first sub-area of the monitored area and a second microphone configured to monitor a second sub-area of the monitored area. The first and second sub-area at least partially overlap, and wherein the data processing system is configured to receive first one or more signals indicative of recorded sounds in the first sub-area from the first microphone and second one or more signals indicative of recorded sounds in the second sub-area from the second microphone, and to determine, based on the first and second one or more signals, a value of a pollination quality parameter indicative of how well one or more flowers in a region of the monitored area, said region comprising said at least partial overlap between the first and second sub-area, are pollinated.

In an embodiment, the data processing system may be configured to determine a particular pollination event in the overlap between the first and second sub-area and/or a duration of a particular pollination event in the overlap between the first and second sub-area based on the first one or more signals and on the second one or more signals. In this embodiment, the particular pollination event left a footprint in both the first one or more signals and in the second one or more signals, i.e. the sound associated with the particular pollination event was recorded by both the first and the second microphone.

These embodiments enable to accurately determine pollination quality parameters for regions that are near the boundaries of sub-areas as monitored by microphones. Sounds produced near a boundary of a particular microphone's sub-area are of course less clearly recorded by the particular microphone than sounds that are produced near that particular microphone. In this embodiment, the sub-areas of the first and second microphone overlap, meaning that the overlapping region is monitored by both the first and second microphone. Sounds produced in the overlapping region are both recorded by the first microphone and by the second microphone. As a result, a sound, for example a sonication sound described above, that is produced in the overlapping region is indicated both by the first one or more signals and by the second one or more signals. This sound will thus have a footprint, though it may be a weak footprint, in both the first one or more signals and in the second one or more signals. The data processing system may be configured to link the two footprints to each other based on their time of occurrence and to, based on this link, determine that the sound was produced in the overlapping region of the first and second sub-area. In particular, the data processing system be configured to, based on this link, determine that a pollination event has occurred in the overlapping region.

In an embodiment, wherein a first and second sub-area at least partially overlap, the data processing system is configured to receive first one or more signals from the first microphone, the first one or more signals indicating a first sound and/or first sound pattern associated with a first pollination event and second one or more signals from the second microphone, the second one or more signals indicating a second sound and/or a second sound pattern associated with a second pollination event. In this embodiment, the data processing system is configured to, based on a similarity of the first sound and second sound and/or based on a similarity of the first sound pattern and second sound pattern, determine that the first and second sound are associated with the same pollination event and/or that the first and second sound pattern are associated with the same pollination event.

This embodiment allows to accurately monitor pollination events in regions where sub-areas overlap. In particular, this embodiment may prevent that the same pollination event is counted more than once.

The similarity may relate to a similarity of time of occurrence, e.g. an extent to which the first and second sound and/or first and second sound patterns were recorded at the same time, and/or a similarity of frequency spectrum, e.g. an extent to which the frequency spectrum of the first and second sounds and/or first and second sound patterns are the same.

In an embodiment, the data processing system is configured to, based on the value of one or more determined pollination quality parameters, estimate a percentage of fertilized flowers.

In an embodiment, the data processing system is further configured to perform a machine learning algorithm for improving the data processing system's capability to determine the value of the pollination quality parameter. Performing the machine learning algorithm comprises receiving training data, the training data comprising a plurality of sets of recorded sounds for respective batches of plants for a certain pollinator type. Each set of recorded sounds is associated in the training data with an actual value for a pollination quality parameter indicative of how well flowers in the associated batch were pollinated. Performing the machine-learning algorithm further comprises building a pollination quality parameter estimation model based on the training data. This may be performed using methods known in the art.

The pollination quality parameter for a batch may for example be an actually measured percentage of fertilized flowers in that batch. The training data may thus be obtained by counting a number of pollination events in a quantified batch of flowers, using one or more microphones for monitoring the area where the batch is present, and then, at an appropriate time, counting the number of flowers that have been properly pollinated. This allows to calculate the percentage of fertilized flowers in the batch. It should be appreciated that the actual yield of a batch, e.g. the amount of eventually harvested fruits, can be regarded as a pollination quality parameter or at least can be taken as input for determining the pollination quality parameter. It is understood that the actual, eventual yield is linked to quality of the pollination. Then, the training data may be construed by storing each set of recorded sounds with the actually measured value for the pollination quality parameter.

Building the pollination quality parameter estimation model based on the training data preferably comprises correlating the recorded sounds with the value of the actual value for the pollination quality parameter.

In an example, this embodiment comprises:
receiving training data comprising a plurality of sets of recorded sounds wherein each set is associated with an area monitored by a microphone during a training period. This area then comprises the batch referred to above.
analysing the recorded sounds,
receiving reported/measured values of a pollination quality parameter for the area during the training period, and
correlating the analysed data with the value of the pollination quality parameter.

In an embodiment, the data processing system is configured to, based on the determined value of the pollination quality parameters for each region of the respective plurality of regions, determine one or more regions of concern out of the plurality of regions, each determined region of concern having a value for the associated pollination quality parameter that is lower than a threshold value.

It should be appreciated that a higher value for the pollination quality parameter indicates that flowers are better pollinated. Thus, this embodiment enables to identify regions in the greenhouse in which, for some reason, the pollination is not performed very well. The greenhouse operator can check these regions and see whether the circumstances are as desired and whether he or she can change anything, for example change the temperature, air flow, lighting, in order to boost the pollination.

In an embodiment, the system comprises a pollination control system configured to influence pollination in selected regions. In this embodiment, the data processing system is configured to, based on the determination of the one or more regions of concern, control the pollination control system to improve the pollination in said one or more regions of concern. The pollination control system is adapted to control an environmental condition in one or more of regions in the monitored area, wherein the environmental condition affects the pollination in the one or more regions. These environmental conditions may comprise a lighting condition, a sound, vibration, air flow, temperature, humidity etc. in the one or more regions. Systems for controlling lighting, sound, vibration, air flow, temperature and humidity are known in the art. For example, near an open window or venting opening in a greenhouse, or at a location of an air inlet of a ventilation system, the air flow might be such that insects are discouraged to fly there and hence pollination is less likely to happen. This is because in general flying insect are disturbed and even disoriented by air flows. (Temporarily) adapting the air flow may thus be used to control pollination in some areas of the greenhouse. Another example of a zone where reduced pollination activity may occur could be a zone that is near a energy aggregate that causes disturbing noise and vibration. Also, a location with intensified illumination or direct line-of-sight with light coming into the greenhouse and towards the plants that is intensified by, e.g., a reflection on the glass panels of the greenhouse or reflective assets as used in the greenhouse may cause reduced pollination activity. Further, also non-uniform temperature and CO2 concentration across the greenhouse may alter pollination uniformity and hence there may always be spots/areas within the greenhouse that provide a less attractive climate condition for the pollinators. Hence, temperature and CO2 control may be used affect pollination in some spots/areas of the greenhouse.

This embodiment enables that a batch of flowers is pollinated uniformly across a greenhouse and that local reductions of the yield due to bad pollination are prevented.

If the pollination control system is used to improve pollination it may also be referred to as a pollination boosting system. The pollination control system may in particular be configured to improve and/or deteriorate the pollination in selected regions.

The data processing system may be configured to, based on the determination of the one or more regions of concern, control the pollination control system to deteriorate the pollination in regions other than the regions of concern. This may be beneficial because a uniform pollination can be achieved. To illustrate, the pollination may influence the time to harvest, and by suppressing the pollination in selected regions, it may be ensured that the fruits are ready for harvest at roughly the same time.

In an embodiment, the pollination control system comprises a horticulture illumination system that is configured to generate pollination light suitable for influencing pollination. The data processing system is then configured to, based on the determination of the one or more regions of concern, control the horticulture illumination system to provide pollination light in said one or more regions of concern for improving the pollination in said one or more regions of concern. This embodiment provides a convenient manner for boosting the pollination in selected regions. The pollination light can for example be such that it attracts pollinators.

The horticulture illumination system may thus be configured to generate pollination light suitable for improving and/or deteriorating pollination. The technologies, in particular the light spectra, disclosed in WO 2015/113818 A1 can be used to attract pollinators and/or to guide them to the regions where pollination should be improved. Generally, a tuneable spectrum, polarization, intensity and/or flickering pattern of light may be used to control the attracting or repelling of pollinators to or from a location. Blue light (e.g., wavelengths around 400-405 nm) or long UVA wavelengths work fine for attracting bumble bees. Also ‘bee's purple’ which is a combination of yellow and ultraviolet attract bumble bees. Additionally or alternatively, an irradiation angle of the flowers may be changed so as to make the flower petals more visible for the bees. Some flower petals appear to change colour, depending upon illumination angle and/or viewing angle. This is known as iridescence. The colour change is often in the UV spectrum and is therefore visible to bees. They can see these shiny petals and associate them with sugar. Thus, the flower becomes more attractive to the bee and gets pollinated.

In an embodiment, the pollination control system comprises a sound producing system configured to produce acoustic signals suitable for influencing pollination. Then, the data processing system is configured to, based on the determination of the one or more regions of concern, control the sound producing system to provide acoustic signals in said one or more regions of concern for improving pollination in said one or more regions of concern.

The sound producing system may thus be configured to produce acoustic signals suitable for improving and/or deteriorating pollination. For example, the sound of a pollinator, such as a bumble bee, flying or a sonication sound may be used to attract additional pollinators. The sound producing system may also apply a self-learning process to listen/record sounds of pollination and reproduce sounds that are associated with successful pollination.

A distinct aspect of this disclosure relates to a method for monitoring pollination of plants carrying one or more flowers. The method comprises receiving, from each of a plurality of microphones for monitoring an area comprising said one or more flowers, one or more signals indicative of recorded sounds. Each microphone out of the plurality of microphones is configured to monitor a sub-area of the monitored area. Further, each microphone is suitable for recording sounds produced by a pollinator, such as a bumblebee, that is present in the microphone's sub-area. The method further comprises determining, based on the one or more signals received from the plurality of microphones, a value of a pollination quality parameter indicative of how well one or more flowers in the monitored area are pollinated. Optionally, this method is computer-implemented. Each step of any method described herein may or may not be computer-implemented.

Further, this method for monitoring pollination may comprise any of the steps that the data processing system described in this disclosure is configured to perform. It should be appreciated that these steps are not necessarily performed by a data processing system. To illustrate, a step of controlling a pollination control system may be performed by a data processing system as well as by a human greenhouse operator.

In an embodiment, the method comprises determining, based on the one or more signals received from the plurality of microphones, for each region out of a plurality of regions in the monitored area, a pollination quality parameter indicative of how well one or more flowers in the region are pollinated. This embodiment also comprises, based on the determined value of the pollination quality parameter for each region of the plurality of regions, determining one or more regions of concern out of the plurality of regions, each region of concern having a value for the associated pollination quality parameters that is lower than a threshold value. This embodiment further comprises, based on the determination of the one or more regions of concern, controlling a pollination control system, configured to influence pollination in selected regions, to improve the pollination in said one or more regions of concern.

The latter step is for example performed by a greenhouse operator controlling a horticulture illumination system and/or sound producing system described herein. Alternatively, this step may be performed by a data processing system. In the latter case, the data processing system may perform this step autonomously in the sense that no human intervention is required.

One aspect of this disclosure relates to a data processing system for use in any system for monitoring pollination of plants as described herein. Such data processing system may be configured to perform any of the methods described herein for monitoring the pollination of plants.

One aspect of this disclosure relates to a computer program comprising instructions which, when executed by a data processing system, cause the data processing system to perform any of the methods described herein for monitoring pollination of plants, including such methods wherein a pollination control system is controlled to influence the pollination.

One aspect of this disclosure relates to a non-transitory computer-readable storage medium storing any of the computer programs described herein.

One aspect of this disclosure relates to a computer comprising a computer readable storage medium having computer readable program code embodied therewith, and a processor, preferably a microprocessor, coupled to the computer readable storage medium, wherein responsive to executing the computer readable program code, the processor is configured to perform any of the methods described herein.

One aspect of this disclosure relates to a computer program or suite of computer programs comprising at least one software code portion or a computer program product storing at least one software code portion, the software code portion, when run on a computer system, being configured for executing any of the methods described herein.

As will be appreciated by one skilled in the art, aspects of the present invention may be embodied as a system, a method or a computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Functions described in this disclosure may be implemented as an algorithm executed by a processor/microprocessor of a computer. Furthermore, aspects of the present invention may take the form of a computer program product embodied in one or more computer readable medium(s) having computer readable program code embodied, e.g., stored, thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples of a computer readable storage medium may include, but are not limited to, the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of the present invention, a computer readable storage medium may be any tangible medium that can contain, or store, a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber, cable, RF, etc., or any suitable combination of the foregoing. Computer program code for carrying out operations for aspects of the present invention may be written in any combination of one or more programming languages, including an object oriented programming language such as Java™, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems), and computer program products according to embodiments of the present invention. It will be understood that each block of the flowchart illustrations and/or block diagrams, and combinations of blocks in the flowchart illustrations and/or block diagrams, can be implemented by computer program instructions. These computer program instructions may be provided to a processor, in particular a microprocessor or a central processing unit (CPU), of a general purpose computer, special purpose computer, or other programmable data processing apparatus to produce a machine, such that the instructions, which execute via the processor of the computer, other programmable data processing apparatus, or other devices create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts, or combinations of special purpose hardware and computer instructions.

Moreover, a computer program for carrying out the methods described herein, as well as a non-transitory computer readable storage-medium storing the computer program are provided. A computer program may, for example, be downloaded (updated) to the existing data processing systems or be stored upon manufacturing of these systems.

Elements and aspects discussed for or in relation with a particular embodiment may be suitably combined with elements and aspects of other embodiments, unless explicitly stated otherwise. Embodiments of the present invention will be further illustrated with reference to the attached drawings, which schematically will show embodiments according to the invention. It will be understood that the present invention is not in any way restricted to these specific embodiments.

### BRIEF DESCRIPTION OF THE DRAWINGS

Aspects of the invention will be explained in greater detail by reference to exemplary embodiments shown in the drawings, in which:

FIG. 1 schematically shows an embodiment of the system for monitoring pollination of plants;

FIG. 2 represents a heat map indicating respective values for respective regions of the monitored area;

FIG. 3 schematically shows an embodiment of the system comprising a horticulture illumination system;

FIG. 4 schematically shows an embodiment of the system comprising a sound producing system;

FIG. 5 is a flow chart illustrating a method according to an embodiment;

FIG. 6 is a flow chart illustrating a machine learning algorithm for building a pollination quality parameter estimation model;

FIG. 7 illustrates a data processing system according to an embodiment.

### DETAILED DESCRIPTION OF THE DRAWINGS

In the figures, identical reference numbers indicate identical or similar elements.

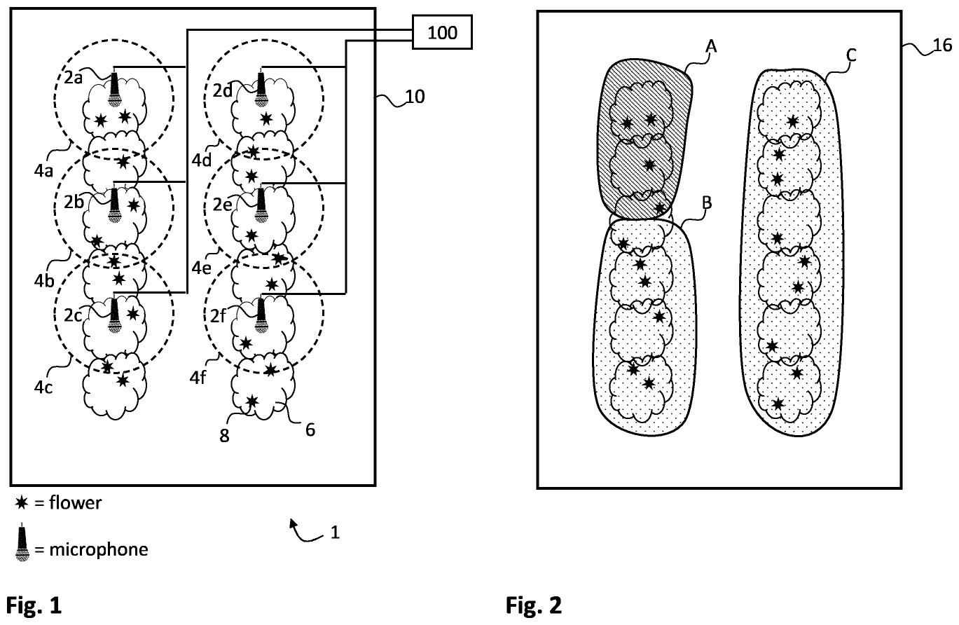

FIG. 1 is a schematically representation of an embodiment of the system 1 for monitoring plants disclosed herein. In particular, FIG. 1 shows a greenhouse 10 in which several plants 6 are present carrying one or more flowers 8. The plants can be any type of plants, as long as they are plants that can be pollinated by pollinators. The plants are for example fruit plants, such as grapevines, blueberry plants, strawberry plants, raspberry plants, blackberry plants, apple trees, cherry trees, peach trees, et cetera, and vegetable plants, such as cucumber plants, tomato plants, eggplants, pepper plants, et cetera.

The system comprises a plurality of microphones 2a-2f for monitoring the area inside the greenhouse 10. The microphones may be installed in existing luminaires, or at least at the position of existing luminaires. The microphones may hang above the plants. Additionally or alternatively, the microphones may be positioned in between the plants. Each microphone is configured to monitor a sub-area of the monitored area. To illustrate, microphone 2a is configured to monitor sub-area 4a, microphone 2b is configured to monitor sub-area 4b et cetera. Each microphone can record sounds produced by a pollinator, such as a bumblebee, that is present in the microphone's sub-area. Further, each microphone can output one or more signals indicative of recorded sounds. These signals can be output to data processing system 100 via respective communication connections between the microphones and the data processing system 100. In the figure, solid lines to and from the data processing system 100 indicate such communication connections. Each communication connection referred to herein may be a wired connection, a wireless connection, or a connection that is partially a wired connection and partially a wireless connection. In an example, the microphones can connect to a packet switched network such as the internet and communicate with the data processing system 100 through the internet. The data processing system is for example a remote server.

The data processing system 100 may be a distributed system, for example in the sense that some elements, such as memory elements, may be present at one or more of the microphones whereas other elements, such as microprocessor, sits remote from the microphones, e.g. at a remote server.

In any case, the data processing system 100 is configured to receive, via one or more input interfaces 112 of the data processing system, from each of the plurality of microphones, the one or more signals indicative of recorded sounds. The data processing system 100 is further configured to, based on the signals received from the plurality of microphones, determine, using at least one processor 102, a value of a pollination quality parameter indicative of how well one or more flowers in the monitored area are pollinated by pollinators present in the monitored area. The data processing system 100 may for example be configured to count a number of pollination events in the monitored area or in a region of the monitored area and/or a duration of each of the pollination events in the monitored area or in a region of the monitored area. As used herein a pollination event may be understood to comprise a pollinator visiting a flower and pollinating the flower. The data processing system 100 may for example count, for a specific region of the monitored area and/or for the entire monitored area as a whole, the average number of pollination events per flower per unit of time, e.g. per day. Based on the number of pollination events and/or based on their measured durations, the value of the pollination quality parameter can be determined.

FIG. 1 shows that the sub-areas 4 that are monitored by the microphones overlap. However, this is not per se required. Some sub-areas may overlap, whereas other may not overlap with any other sub-area. Also, embodiments are envisaged wherein none of the sub-areas 4 overlap with another sub-area 4. It should be appreciated that if two sub-areas at least partially overlap, then their two associated microphones can record sounds of a pollinator that is present in the region where the two sub-areas overlap. To illustrate, sub-areas 4a and 4b at least partially overlap. This means that microphone 2a and microphone 2b will record sounds as produced by a pollinator that is present in this overlapping region. The data processing system 100 may then be configured to determine a value for a pollination quality parameter indicative of how well flowers in the overlap between the sub-area 4a and sub-area 4b are pollinated, based on both signals from microphone 2a and on signals from microphone 2b. By having multiple microphones cover the same area, the accuracy with which pollination events can be detected in this area can be improved. Further, this also allows to localize measured pollination events better. After all, if the same pollination event has a footprint in both the sounds as recorded by microphone 2a and in the sounds as recorded by microphone 2b, then this pollination event must have occurred in the overlap between sub-area 2a and sub-area 2b. The overlapping region of two microphones may be relatively large.

In an embodiment, the plurality of microphones may comprise one or more microphone arrays known in the art, wherein each microphone in a microphone array may monitor substantially the same area. This enables to retrieve the direction of a sound and hence to determine the location of a recorded sound. This has the advantage that a large area can be monitored and still location information from the recorded sounds can be determined. The microphone array can also be used for beam forming to improve the signal to noise ratio by removing noise sources. Using a microphone array enables more precise localization of pollination events and also give an indication of trajectory in flight of pollinators.

The data processing system 100 may be configured to determine for multiple regions a respective pollination quality parameter. It should be appreciated that the regions for which a value of a pollination quality parameter is determined, do not necessarily coincide with the sub-areas 4 of the microphones 2. In an embodiment, however, each region corresponds to a sub-area. Then, the data processing system 100 effectively determines for each sub-area, a value for a pollination quality parameter indicative of how well one or more flowers are pollinated in the sub-area in question.

The data processing system 100 may further be configured to, via one or more output interfaces 114, render on a display 16 the regions and their respective values for the respective pollination quality parameters. This may be rendered in the form of a heat map.

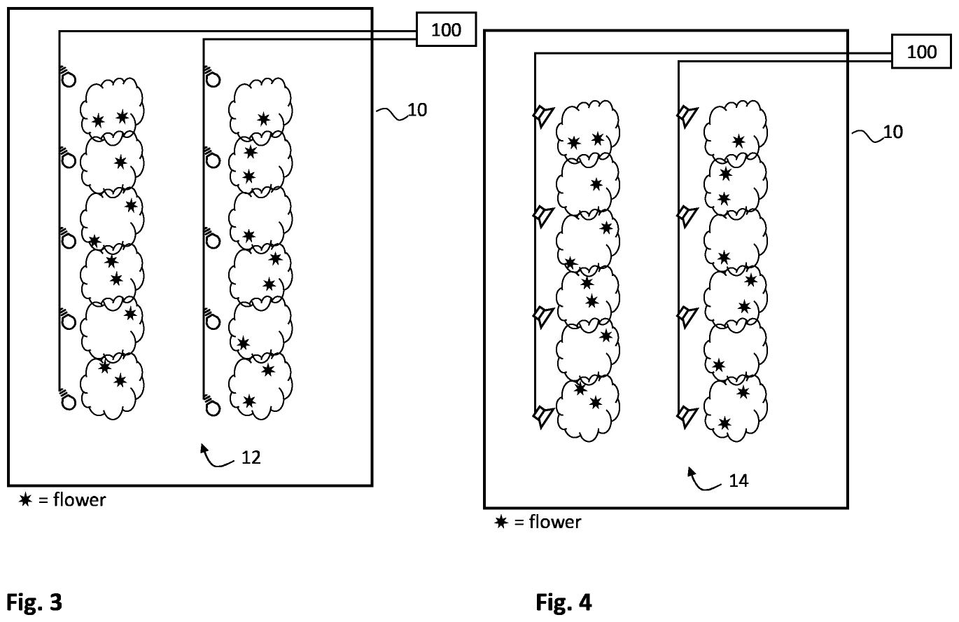

FIG. 2 illustrates an example of a display 16 presenting pollination quality parameters per region. Display 16 presents three region A, B, C. Regions B and C have similarly valued pollination quality parameters. In this example, it is assumed that the flowers in regions B and C are pollinated well. However, the pollination quality parameter for region A has a relatively low value, i.e. relatively low with respect to the values of regions B and C. This lower value indicates that the flowers in region A are pollinated not as well as the flowers in regions B and C. A greenhouse operator who sees this heat map, can go and inspect region A in order to see if the circumstances in region A are as they should be and to see whether he or she can take measures to improve the pollination in this region A. Circumstances may refer to environmental conditions such as light conditions, sound, vibrations, air flow, temperature, humidity etc. and hence the greenhouse operator may take measures to change one or more of these environmental conditions to improve pollination.

Region A may thus be determined to be a region of concern as referred to above having a value for the pollination quality parameter that is lower than a threshold value. Which threshold values to use greatly depends on the case at hand (which plants, which pollinators, et cetera), however, suitable threshold values can be determined for any situation.

FIG. 3 schematically shows an embodiment wherein the system comprises a pollination control system configured to influence pollination in selected regions. In this embodiment, the system also comprises the plurality of microphones, however, these are not shown in FIG. 2 for clarity. The data processing system 100 is configured to, based on the determination of one or more regions of concern, control the pollination control system to improve the pollination in said one or more regions of concern. In FIG. 3, the pollination control system comprises a horticulture illumination system 12 that is configured to generate pollination light suitable for influencing pollination. As explained above, the horticulture illumination system can be controlled to provide pollination light in the one or more regions of concern for improving the pollination in said one or more regions of concern.

FIG. 4 schematically shows an embodiment of the system that also comprise a pollination control system embodied as a sound producing system 14 configured to produce acoustic signals suitable for influencing pollination. This embodiment also comprises the plurality of microphones. These are not shown for clarity. The sound producing system 14 preferably comprises a plurality of microphones. These microphones may then be positioned at different locations throughout the monitored area, so that acoustic signals can be provided selectively in different regions, preferably of course the identified regions of concern. The data processing system 100 is configured to, based on the determination of the one or more regions of concern, control the sound producing system to provide acoustic signals in said one or more regions of concern for improving pollination in said one or more regions of concern.

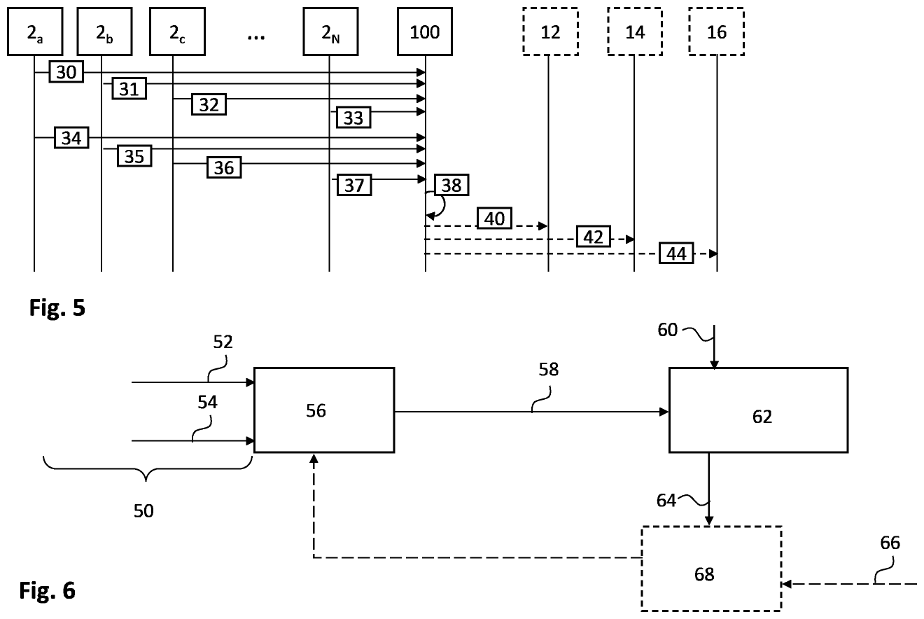

FIG. 5 is a flow chart illustrating an embodiment of the method for monitoring pollination of plants. In steps 30-37, the microphones 2ₐ-2_{N} send signals to the data processing system 100. The data processing system 100 thus receives from each microphone one or more signals. These signals indicate sounds as recorded by the microphones.

In step 38, the data processing system 100 determines, based on the one or more signals received in steps 30-37, a value of a pollination quality parameter indicative of how well one or more flowers in the monitored area are pollinated. This step may be embodied as the data processing system 100 determining a value for an overall pollination quality parameter indicating how well flowers are pollinated in the entire area monitored by the plurality of microphones. Additionally or alternatively, this step may be embodied as the data processing system 100 determining a value for a specific region or as the data processing system 100 determining respective values for respective regions in the monitored area.

The data processing system may be configured to determine a pollination event by recognizing from the one or more signals from the plurality of microphones, a sound that is associated with a pollination event and/or a sound pattern that is associated with a pollination event. The spectrograph shown in FIG. 1 of Predicting species identity of bumblebees through analysis of flight buzzing sounds by Gradišek et al, The International Journal of Animal Sound and its Recording, Volume 26, 2017—Issue 1, pages 63-76, May 2016 (hereinafter referred to as “Gradišek”) shows the frequency spectrum of a sonication sound, which is a sound that is associated with a pollination event. The data processing system 100 may thus be configured to recognize the sound, by recognizing a frequency spectrum of the sound.

In an embodiment, wherein the data processing system is configured to recognize a sound pattern, the sound pattern may be understood to be a time-lapsed sound pattern and comprises a first time period comprising a sound associated with flying of a pollinator, a subsequent second time period substantially without sounds associated with flying of a pollinator and a subsequent third time period comprising a sound associated with flying of a pollinator. It is noted that FIG. 1 of Gradišek also shows a sound pattern associated with a pollination event in that it shows between 0 and 10 seconds sounds associated with flying of a bee, between 10 and 15.5 seconds sounds substantially without sounds associated with flying of a pollinator and then again from 15.5 seconds onwards sounds associated with flying of a pollinator.

A bee, such as a bumblebee, pollinating a flower typically performs a sequence of actions described below. Based on these actions and their associated sounds, a sound pattern can be recognized in recorded sounds.
Approach towards a flower: The microphone (that is either localized near a flower or directed towards a flower) captures the sound of the bumble bee in its flight mode (with a distinct wing frequency). The approach can be deduced from the increasing amplitude of the sound.
Landing onto a flower: At the event of landing, the sound ‘disappears’ all at once (as the bumble bee has its wings immobilized). This is the starting point for ‘measuring’ the residence time of the bumble bee on the flower.
Residing on a flower: The period that the bumble bee is residing on the flower (interacting, collecting nectar and pollen) is the time that the bumble bee is silent (this is the time between landing and lift-off) or the time that a sonication sound or sonication sound pattern is produced by the bumble bee. This time is important because a long enough time means that the bee has a ‘rewarding’ interaction with the flower (feasting on the feed it finds) and hence most likely fertilizes the flower because of entering deep into the flower with high likelihood of fertilization.
Lift-off from a flower: The lift-off can be detected by the start of sound again. This could be characterized by not only the onset of sound, but also by an increasing frequency and amplitude of the sound.
Flying away: The fly away event is comparable to the approach phase, but the opposite. Lift-off is first detected, and next the bumble bee goes into a fixed wing frequency mode and the amplitude goes down when the distance from the bumble bee to the microphone increases.
Communication signal: When the bumble bee is generating sounds to for example recruit others, the likelihood of pollination increases. One reason is that the bee indicates flower is a decent food source which it will visit. Also, it recruits more pollinators increasing the chance that the flower will be visited by more pollinators.

Optionally, as indicated by the dashed lines, the data processing system 100 controls the horticulture illumination system 12 to locally produce pollination light in order to improve the pollination in selected regions (step 40) and/or the sound producing system 14 to locally provide acoustic signals in order to improve the pollination in selected regions (step 42) and/or the display 16 in order to render one or more determined pollination quality parameter values for one or more respective regions of the monitored area on a display, optionally in the form of a heat map (step 44).

FIG. 6 is a flow chart illustrating an embodiment of the method where a machine learning algorithm is performed for improving the data processing system's capability to determine the value of a pollination quality parameter based on recorded sounds.

Initially, training data 50 are obtained. These training data 50 comprise a plurality of sets 52 of recorded sounds for respective batches of plants, wherein each set of recorded sounds is associated in the training data 50 with an actual value 54 for a pollination quality parameter indicative of how well flowers in the associated batch were pollinated.

These training data 50 are then used in step 56 to correlate pollination quality values to recorded sounds. Step 56 thus comprises correlating pollination quality values to recorded sounds. Output of this step 56 is a pollination quality parameter estimation model 58, which is used in step 62 to, based on recorded sounds 60, determine one or more values 64 of pollination quality parameters. Output of step 62 is a set 64 of one or more values for respective on or more pollination quality parameters.

Optionally, as indicated by the dashed lines, a step 68 is performed. In this step 68, the values of the pollination quality parameters as determined in step 62 are compared to actually measured values 66 of the pollination quality parameters to see to what extent the determined pollination quality parameters 64 were correct. Of course, the values determined in step 62 and the actually measured values 66 used for the comparison in step 68 are for the same respective batches of plants. The actually measured values 66 and recorded sounds 60 may be subsequently used as training data in order to improve the correlation, i.e. in order to improve the pollination quality parameter estimation model that is used in step 62.

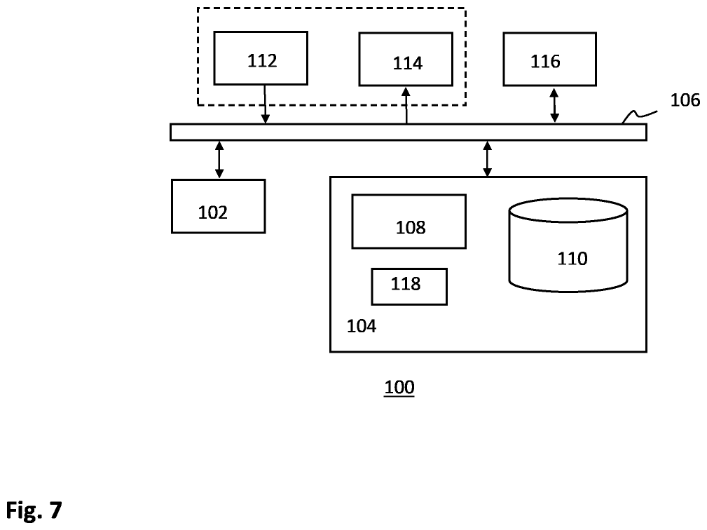

FIG. 7 depicts a block diagram illustrating a data processing system according to an embodiment. In general, the data processing system may also be referred to herein as a data processor, a data processing unit, a data processing server or a data processing computer.

As shown in FIG. 7, the data processing system 100 may include at least one processor 102 coupled to memory elements 104 through a system bus 106. As such, the data processing system may store program code within memory elements 104. Further, the processor 102 may execute the program code accessed from the memory elements 104 via a system bus 106. In one aspect, the data processing system may be implemented as a computer that is suitable for storing and/or executing program code. It should be appreciated, however, that the data processing system 100 may be implemented in the form of any system including a processor and a memory that is capable of performing the functions described within this specification.

The memory elements 104 may include one or more physical memory devices such as, for example, local memory 108 and one or more bulk storage devices 110. The local memory may refer to random access memory or other non-persistent memory device(s) generally used during actual execution of the program code. A bulk storage device may be implemented as a hard drive or other persistent data storage device. The processing system 100 may also include one or more cache memories (not shown) that provide temporary storage of at least some program code in order to reduce the number of times program code must be retrieved from the bulk storage device 110 during execution.

Input/output (I/O) devices depicted as an input device 112 and an output device 114 optionally can be coupled to the data processing system. Examples of input devices may include, but are not limited to, the plurality of microphones referred to herein, a keyboard, a pointing device such as a mouse, a touch-sensitive display, or the like. Examples of output devices may include, but are not limited to, the display 16 referred to herein, the pollination control system referred to herein, such as the horticulture illumination system referred to herein and/or the sound producing system referred to herein, or the like. Input and/or output devices may be coupled to the data processing system either directly or through intervening I/O controllers.

In an embodiment, the input and the output devices may be implemented as a combined input/output device (illustrated in FIG. 7 with a dashed line surrounding the input device 112 and the output device 114). An example of such a combined device is a touch sensitive display, also sometimes referred to as a “touch screen display” or simply “touch screen”. In such an embodiment, input to the device may be provided by a movement of a physical object, such as e.g. a stylus or a finger of a user, on or near the touch screen display.

A network adapter 116 may also be coupled to the data processing system to enable it to become coupled to other systems, computer systems, remote network devices, and/or remote storage devices through intervening private or public networks. The network adapter may comprise a data receiver for receiving data that is transmitted by said systems, devices and/or networks to the data processing system 100, and a data transmitter for transmitting data from the data processing system 100 to said systems, devices and/or networks. Modems, cable modems, and Ethernet cards are examples of different types of network adapter that may be used with the data processing system 100.

As pictured in FIG. 7, the memory elements 104 may store an application 118. In various embodiments, the application 118 may be stored in the local memory 108, the one or more bulk storage devices 110, or apart from the local memory and the bulk storage devices. It should be appreciated that the data processing system 100 may further execute an operating system (not shown in FIG. 7) that can facilitate execution of the application 118. The application 118, being implemented in the form of executable program code, can be executed by the data processing system 100, e.g., by the processor 102. Responsive to executing the application, the data processing system 100 may be configured to perform one or more operations or method steps described herein.

In an aspect, the data processing system 100 may represent a server. For example, the data processing system may represent an (HTTP) server, in which case the application 118, when executed, may configure the data processing system to perform (HTTP) server operations.

Various embodiments of the invention may be implemented as a program product for use with a computer system, where the program(s) of the program product define functions of the embodiments (including the methods described herein). In one embodiment, the program(s) can be contained on a variety of non-transitory computer-readable storage media, where, as used herein, the expression “non-transitory computer readable storage media” comprises all computer-readable media, with the sole exception being a transitory, propagating signal. In another embodiment, the program(s) can be contained on a variety of transitory computer-readable storage media. Illustrative computer-readable storage media include, but are not limited to: (i) non-writable storage media (e.g., read-only memory devices within a computer such as CD-ROM disks readable by a CD-ROM drive, ROM chips or any type of solid-state non-volatile semiconductor memory) on which information is permanently stored; and (ii) writable storage media (e.g., flash memory, floppy disks within a diskette drive or hard-disk drive or any type of solid-state random-access semiconductor memory) on which alterable information is stored. The computer program may be run on the processor 102 described herein.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of embodiments of the present invention has been presented for purposes of illustration but is not intended to be exhaustive or limited to the implementations in the form disclosed. Many modifications and variations will be apparent to those of ordinary skill in the art. The embodiments were chosen and described in order to best explain the principles and some practical applications of the present invention, and to enable others of ordinary skill in the art to understand the present invention for various embodiments with various modifications as are suited to the particular use contemplated.

1. A system for monitoring pollination of plants carrying one or more flowers, the system comprising:
a plurality of microphones for monitoring an area comprising said one or more flowers, wherein each microphone out of the plurality of microphones is configured to monitor a sub-area of the monitored area, wherein each microphone is suitable for recording sounds produced by a pollinator, such as a bumblebee, that is present in the microphone's sub-area, and is configured to output one or more signals indicative of recorded sounds, the system further comprising:
a data processing system comprising at least one input interface and at least one processor that is configured to:
receive, via the at least one interface from each of the plurality of microphones, said one or more signals indicative of recorded sounds, and to
determine, using the at least one processor, based on the one or more signals received from the plurality of microphones, a value of a pollination quality parameter indicative of how well one or more flowers in the monitored area are pollinated, wherein the value of the pollination quality parameter is determined based on a number of pollination events and/or based on a duration of each of the pollination events determined by the data processing system based on the one or more signals received from the plurality of microphones, wherein each pollination event comprises a pollinator visiting a flower and wherein the data processing system is configured to determine a pollination event by recognizing from the one or more signals from the plurality of microphones, a sound that is associated with a pollination event and/or a sound pattern that is associated with a pollination event.

2. The system according to claim 1, wherein the data processing system is configured to:
for each region out of a plurality of regions in the monitored area, determine, based on the one or more signals received from the plurality of microphones, a number of pollination events in the region and/or a duration of each of the pollination events in the region, each pollination event comprising a pollinator visiting a flower, and to
for each region, based on the determined number of pollination events in the region and/or based on the determined durations of the pollination events in the region, determine a value of a pollination quality parameter indicative of how well one or more flowers in the region are pollinated.

3. The system according to claim 2,
wherein the sound pattern is a time-lapsed sound pattern and comprises a first time period comprising a sound associated with flying of a pollinator, a subsequent second time period substantially without sounds associated with flying of a pollinator and a subsequent third time period comprising a sound associated with flying of a pollinator; and
wherein the pollinators are bees, such as bumblebees, and wherein the sound associated with a pollination event is a sonication sound and/or wherein the sound pattern associated with a pollination event is a sonication sound pattern.

4. The system according to claim 1, wherein the plurality of microphones comprises a first microphone configured to monitor a first sub-area of the monitored area and a second microphone configured to monitor a second sub-area of the monitored area, wherein the first and second sub-area at least partially overlap, and wherein the data processing system is configured to:
receive first one or more signals indicative of recorded sounds in the first sub-area from the first microphone and second one or more signals indicative of recorded sounds in the second sub-area from the second microphone, and to
determine, based on the first and the second one or more signals, a value of a pollination quality parameter indicative of how well one or more flowers in a region of the monitored area, said region comprising said at least partial overlap between the first and second sub-area, are pollinated.

5. The system according to claim 1, wherein the data processing system is further configured to perform a machine learning algorithm for improving the data processing system's capability to determine the value of the pollination quality parameter, wherein performing the machine learning algorithm comprises:
receiving training data, the training data comprising a plurality of sets of recorded sounds for respective batches of plants, wherein each set of recorded sounds is associated in the training data with an actual value for a pollination quality parameter indicative of how well flowers in the associated batch were pollinated, and
building a pollination quality parameter estimation model based on the training data.

6. The system according to claim 2, wherein the data processing system is configured to;
based on the determined value of the pollination quality parameter for each region of the plurality of regions, determine one or more regions of concern out of the plurality of regions, each region of concern having a value for the associated pollination quality parameters that is lower than a threshold value.

7. The system according to claim 6, further comprising:
a pollination control system configured to influence pollination in selected regions by controlling one or more environmental conditions selected from a lighting condition, a sound, a vibration, an air flow, a temperature and a humidity in the selected regions, wherein the data processing system is configured to:
based on the determination of the one or more regions of concern, control the pollination control system to improve the pollination in said one or more regions of concern by controlling an environmental condition selected from a lighting condition, a sound, a vibration, an air flow, a temperature and a humidity in one or more of said selected regions.

8. The system according to claim 7, wherein the data processing system is configured to, based on the determination of the one or more regions of concern, control the pollination control system to improve the pollination in said one or more regions of concern by controlling an environmental condition in one or more of said selected regions other than said one or more regions of concern to deteriorate the pollination in said selected regions other than said one or more regions of concern.

9. The system according to claim 7, wherein the data processing system is configured to, based on the determined value of the pollination quality parameter for each region of the plurality of regions, control the pollination control system to control the pollination in the plurality of regions to achieve a substantially uniform pollination across the plurality of regions in the monitored area.

10. The system according to claim 9, wherein the pollination control system comprises a horticulture illumination system that is configured to generate pollination light suitable for influencing pollination, wherein
the data processing system is configured to, based on the determination of the one or more regions of concern, control the horticulture illumination system to provide pollination light in said one or more regions of concern for improving the pollination in said one or more regions of concern,
wherein the pollination light comprises wavelengths of blue and/or long UVA.

11. The system according to claim 9, wherein the pollination control system comprises a sound producing system configured to produce acoustic signals suitable for influencing pollination, wherein
the data processing system is configured to, based on the determination of the one or more regions of concern, control the sound producing system to provide acoustic signals in said one or more regions of concern for improving pollination in said one or more regions of concern,
wherein the acoustic sound signals comprise a sound of a pollinator flying or a sonication sound.

12. A method for monitoring pollination of plants carrying one or more flowers, the method comprising:
receiving, from each of a plurality of microphones for monitoring an area comprising said one or more flowers, one or more signals indicative of recorded sounds, wherein
each microphone out of the plurality of microphones is configured to monitor a sub-area of the monitored area, wherein each microphone is suitable for recording sounds produced by a pollinator, such as a bumblebee, that is present in the microphone's sub-area,
determining, based on the one or more signals received from the plurality of microphones, a number of pollination events and/or a duration of each of the pollination events wherein each pollination event comprises a pollinator visiting a flower, and
determining, based on said number of pollination events and/or a duration of each of the pollination events, a value of a pollination quality parameter indicative of how well one or more flowers in the monitored area are pollinated.

13. The method according to claim 12, further comprising
determining, based on the one or more signals received from the plurality of microphones, for each region out of a plurality of regions in the monitored area, a pollination quality parameter indicative of how well one or more flowers in the region are pollinated, and
based on the determined value of the pollination quality parameter for each region of the plurality of regions, determining one or more regions of concern out of the plurality of regions, each region of concern having a value for the associated pollination quality parameters that is lower than a threshold value, and
based on the determination of the one or more regions of concern, controlling a pollination control system, configured to influence pollination in selected regions by controlling an environmental condition selected from a lighting condition, a sound, a vibration, an air flow, a temperature and a humidity in the selected regions, to improve the pollination in said one or more regions of concern.

14. A data processing system for use in the system for monitoring pollination of plants according to claim 1, the data processing system comprising:
at least one input interface adapted to receive one or more signals indicative of recorded sounds from each of a plurality of microphones, and
at least one processor adapted to determine, based on the one or more signals from the plurality of microphones, a value of a pollination quality parameter indicative of how well one or more flowers in the monitored area are pollinated, wherein the value of the pollination quality parameter is determined based on a number of pollination events and/or based on a duration of each of the pollination events determined by the data processing system based on the one or more signals received from the plurality of microphones, wherein each pollination event comprises a pollinator visiting a flower.

15. A non-transitory computer readable medium comprising instructions which, when executed by at least one processor of the data processing system, cause the data processing system to perform the method according to claim 12.
